(19) United States
(12) Patent Application Publication
(10) Pub. No.: US 2015/0227881 A1
(43) Pub. Date: Aug. 13, 2015

(54) SYSTEM FOR MANAGING AND COORDINATING HEALTH AREA STAFFS THAT WORK UNDER ON-DUTY REGIME AND EQUIPMENT TO IMPLEMENT A SYSTEM FOR MANAGING AND COORDINATING HEALTH AREA STAFFS THAT WORK UNDER ON-DUTY REGIME

(71) Applicant: LEDWARE TECNOLOGIA EM SOFTWARE LTDA., Sao Paulo (BR)

(72) Inventors: VANDERLEI HENRIQUE PEREIRA, Cruzeiro/SP (BR); GUSTAVO NOVAES MARTINS FERREIRA, Sao Paulo (BR)

(73) Assignee: LEDWARE TECNOLOGIA EM SOFTWARE LTDA., Sao Paula (BR)

(21) Appl. No.: 14/176,298

(22) Filed: Feb. 10, 2014

(51) Int. Cl. G06Q 10/06; G06Q 50/22

(52) U.S. Cl. CPC G06Q 10/063118; G06Q 50/22

(57) ABSTRACT

System for managing and coordinating health area staffs that work under on-duty regime and equipment to implement a system for managing and coordinating health area staffs that work under on-duty regime, wherein the present patent purposes a system for managing and coordinating professional staffs, preferably of health area, that work in on-duty regime. The system described herein aims to make more efficient, less arduous and totally transparent the access and participation of professionals that act under on-duty regime in the work scales that are organized for this means. The present patent purposes yet a hardware environment in which this created system is stored for allowing managing and coordinating health area staffs that work in an on-duty regime. The system described herein adopts a computer that is called as server (SERV), in which the software (S) is installed; the server (SERV) is connected to the internet, that constitutes the virtual environment where the system concerned, indicated by reference (SIS) may be accessed, under the many conditions, be it by the Administrator (A), or by Coordinator(s) (C) also and yet by health area professionals—doctors (P).

The present specification relates to an invention patent application that proposes a system for managing and coordinating professional staffs, preferably in the health area, that work in on-duty regime. The system described herein aims to make the access and participation of professionals working under on-duty regime more efficient, less arduous and totally transparent in the work scales organized for this mean. The present patent further proposes a hardware environment in which this created system is housed for allowing managing and coordinating health area staffs that work in on-duty regime.

As is generally known, many professionals particularly in the health area and further more specifically in the medical branch work under on-duty regime, according to which fulfill working hours in specific dates and times aiming this way to maintain the institutions to which their service is offered constantly backed up with professionals of the interest areas.

This type of work under on-duty regime presents some particularities since the scale to which these professionals shall obey needs to be organized in a way to not leave gaps that may represent the absence of a certain professional of a specific area.

Generally, the management and coordination of on-duty staffs, more particularly medical staffs, may be understood through the following description.

Traditional Management/Coordination of Medical Staffs' Process

Many institutions in the health area act with medical staffs taking turns in on-duty scales. In general, there is a medical staff for each specialty, with one or more coordinators in each staff. The coordinators are responsible for the on-duty doctors scale integrity. They offer a service of medical allocation in available time, on-duty changes according to the availability of on-duty doctors, hiring new doctors, replacement in case of time off and covering eventual absences. This function depends on constant information exchange with on-duty doctors. Administering this whole process is tiresome and demands a great amount of time, with misunderstandings being common to take place between on-duty doctors and coordinators.

The constant information change with staff members is performed through conversations during work or by cell phone or email change and SMS or through social network. The scales with all medical on-duty are sent through email to all the staff, generally with spreadsheet in Excel format, being that after some hours they are already outdated due to constant on-duty change and unforeseen actions. It is common that complaints happen by the on-duty doctors due to the difficulty of being in touch with the coordinators and it is not unusual to have misunderstandings regarding to a certain appointed doctor on duty due to misunderstandings in staff information exchange.

In this sense, the current practice can be resumed in three main topics:
Scales sent by email or fixed in a bulletin board in work places.
Difficulty to keep scale updated and available for all staff members.
The on-duty doctor depends on the coordinator confirmation to get an available on-duty site.

In face of the state of the art described above, the process described herein was developed, which aims to allow the introduction of a new concept that covers managing and coordinating health area staffs that work under on-duty regime.

The process proposed herein, although it has been thought and made viable to be used by health area professionals, more particularly in the medical branch, can also has and be alternatively employed to other activity branches that involves work scale under on-duty regime.

The object process of this invention patent application proposes the use of a computer platform, where data referring to on-duty availability, such as dates and times may be accessed and requested by professionals registered to offer this kind of service.

The object process of this invention patent application can be understood through a series of flowcharts that are expressed in the Figs. related bellow, in which:

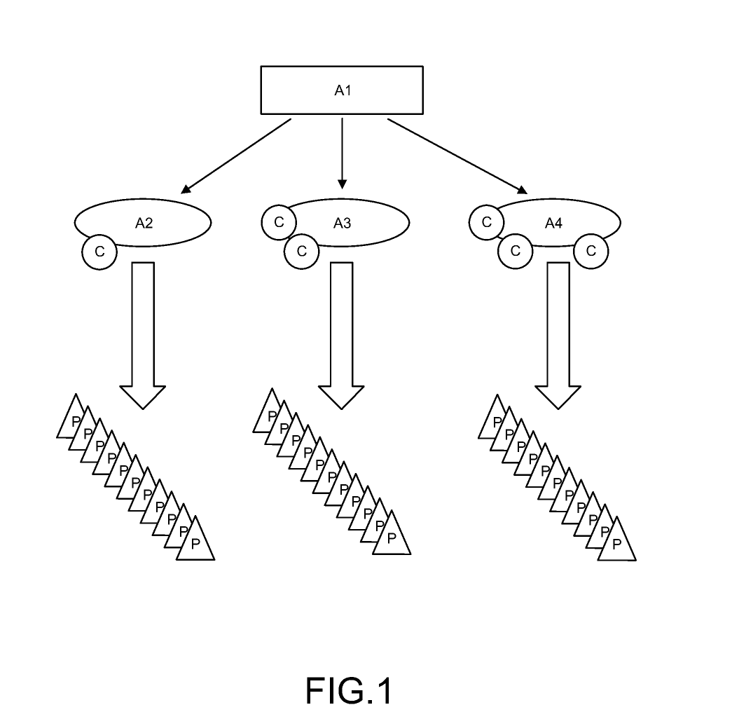

FIG. 1 illustrates a general flowchart relative to the interaction between health service and many management and coordination staffs and its respective professional boards;

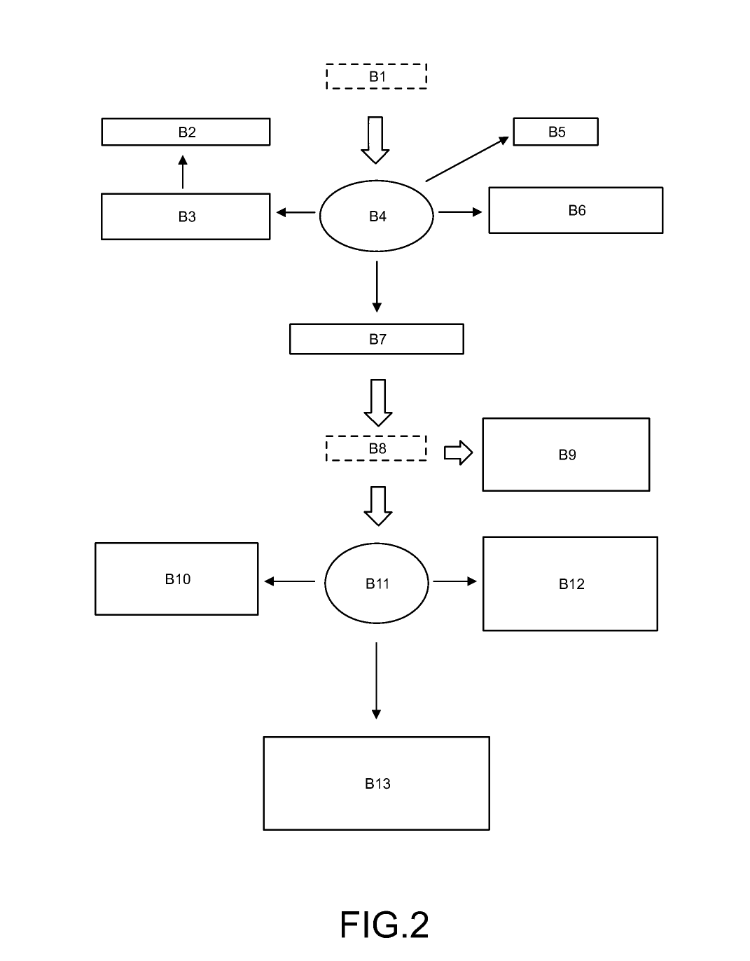

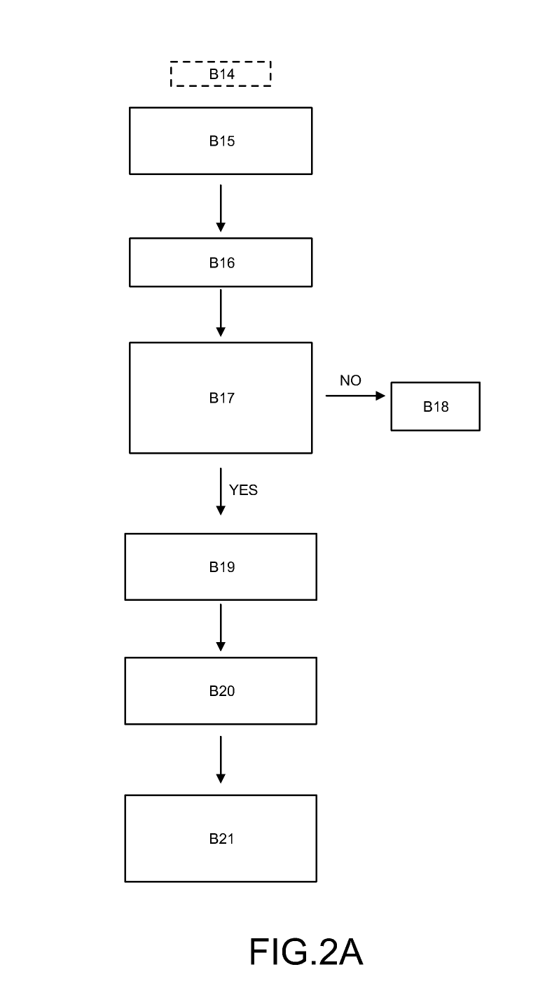

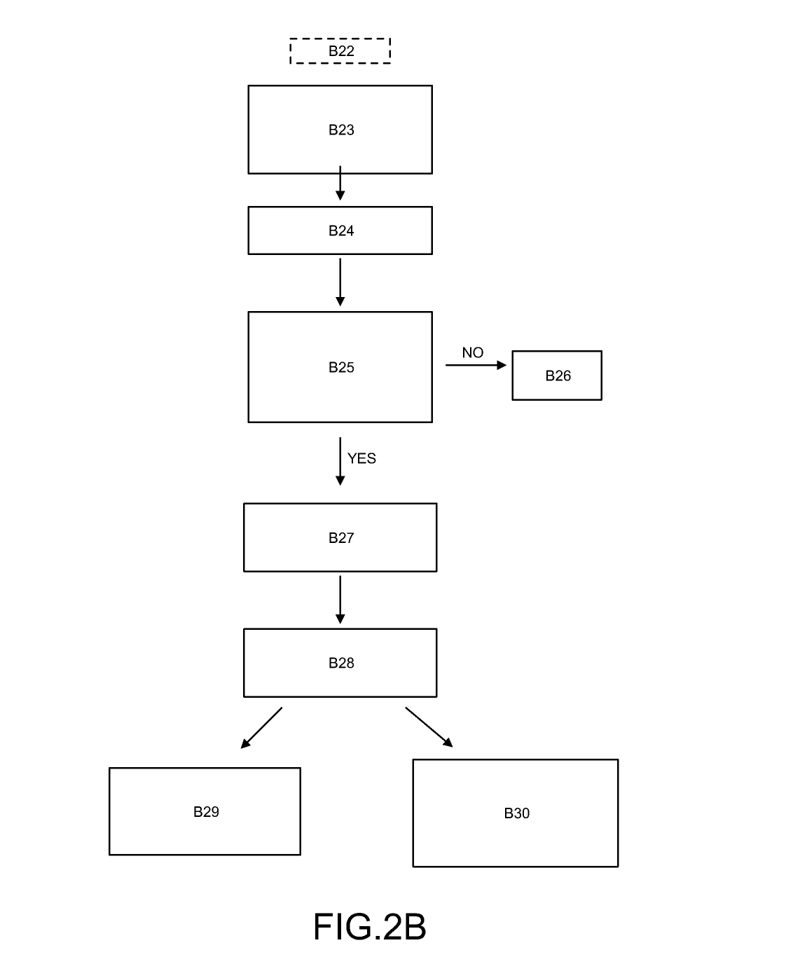

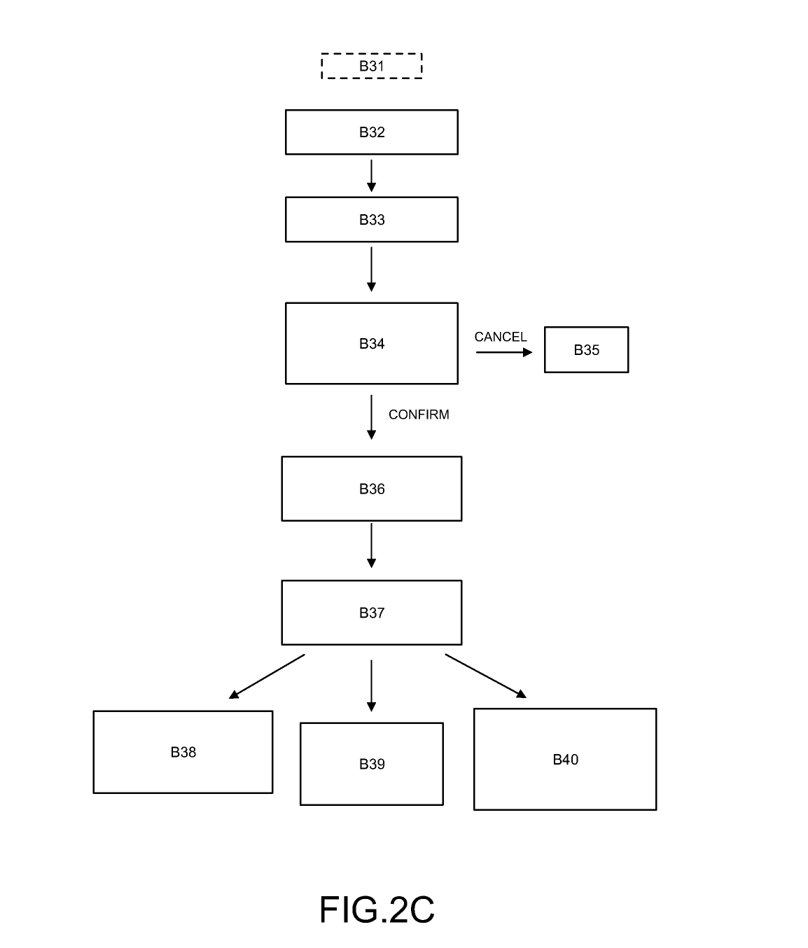

FIG. 2 illustrates a general flowchart relative to the access to the on-duty system from professionals (On-Duty Users);

FIG. 2A illustrates a specific flowchart to one of the Interactive Actions in Scale—To Get On-duty, flowchart that derives from flowchart presented in FIG. 2;

FIG. 2B also illustrates a specific flowchart to one of the Interactive Actions in Scale—To Make Available On-duty, flowchart that also derives from flowchart presented in FIG. 2;

FIG. 2C also illustrates a specific flowchart to one another of the Interactive Actions in Scale—Application Changes, flowchart that is equally derived from flowchart presented in FIG. 2;

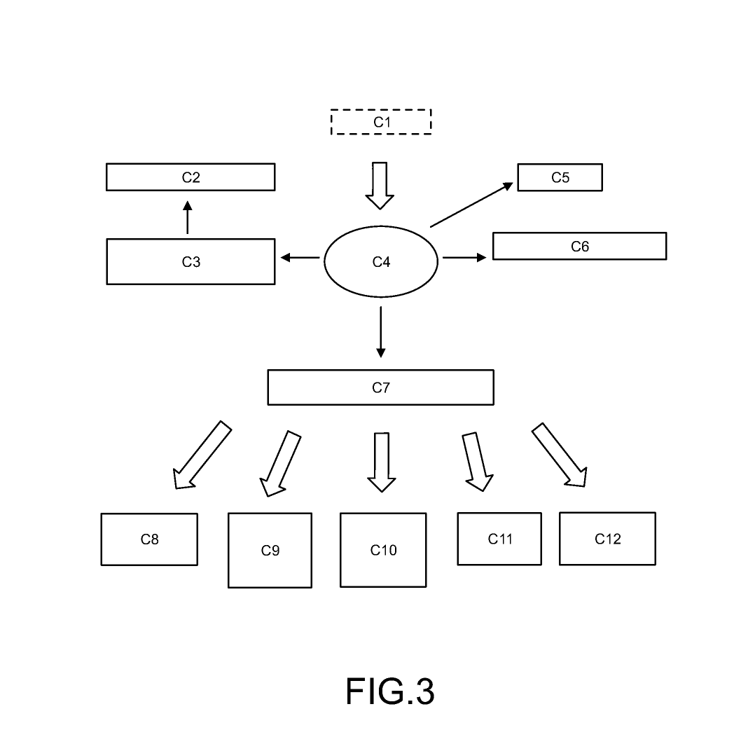

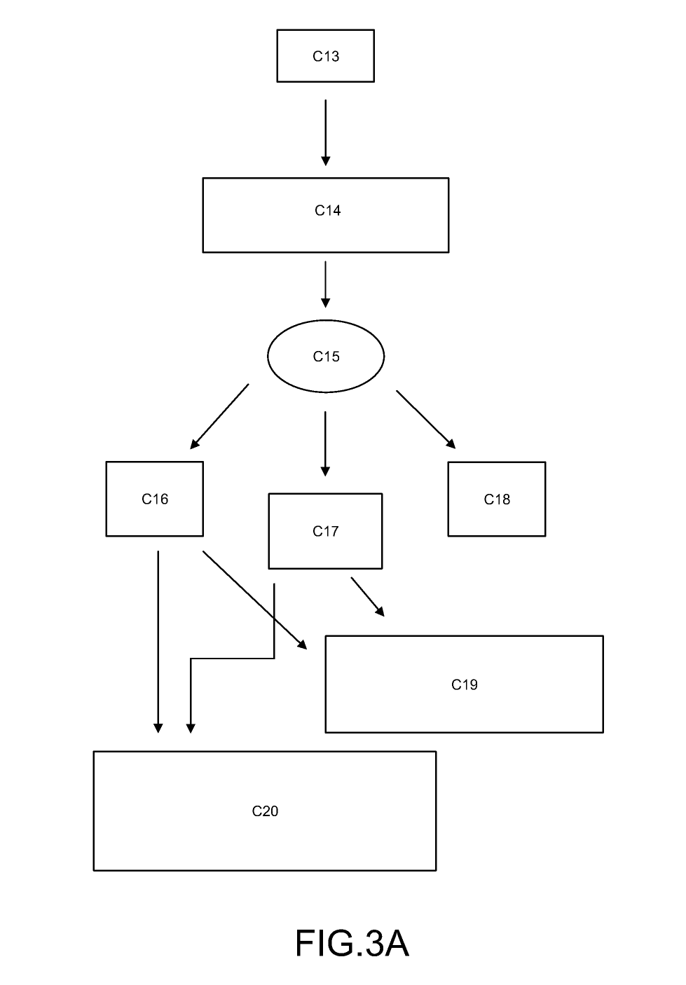

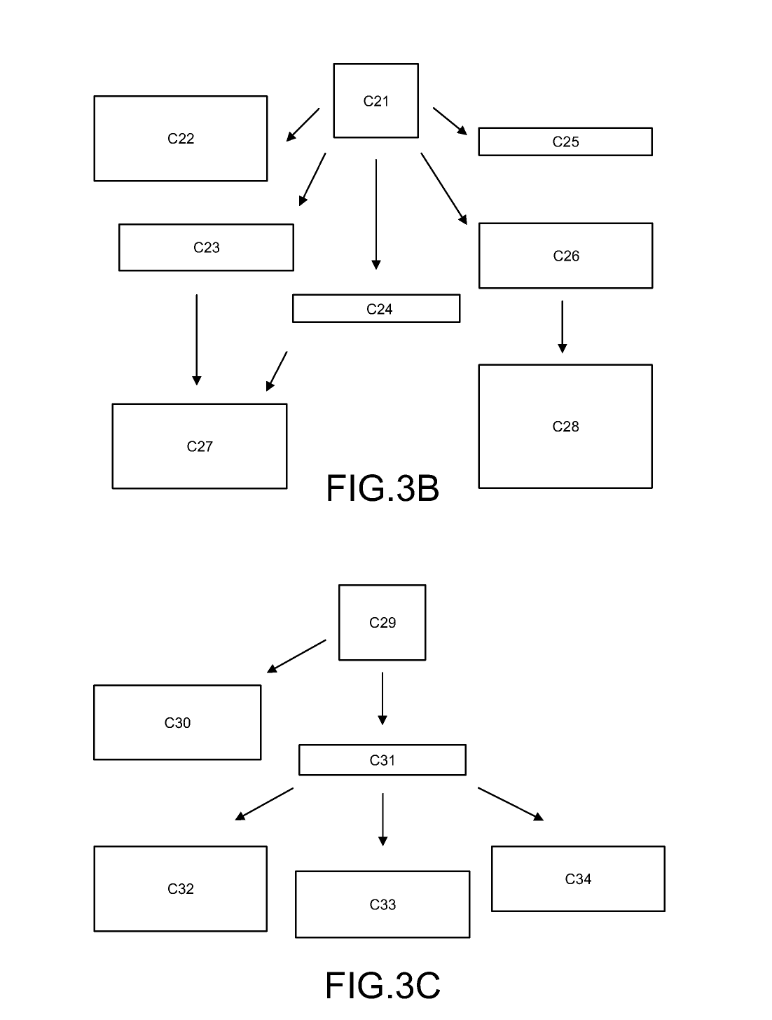

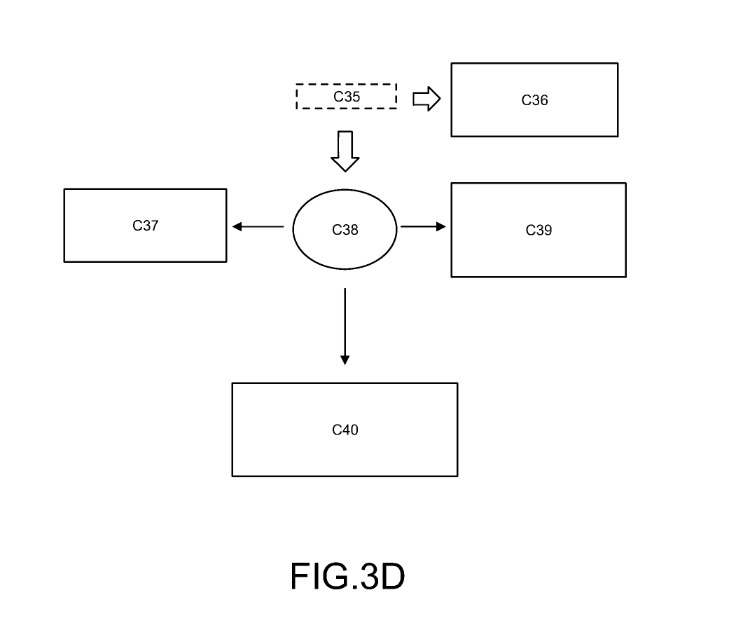

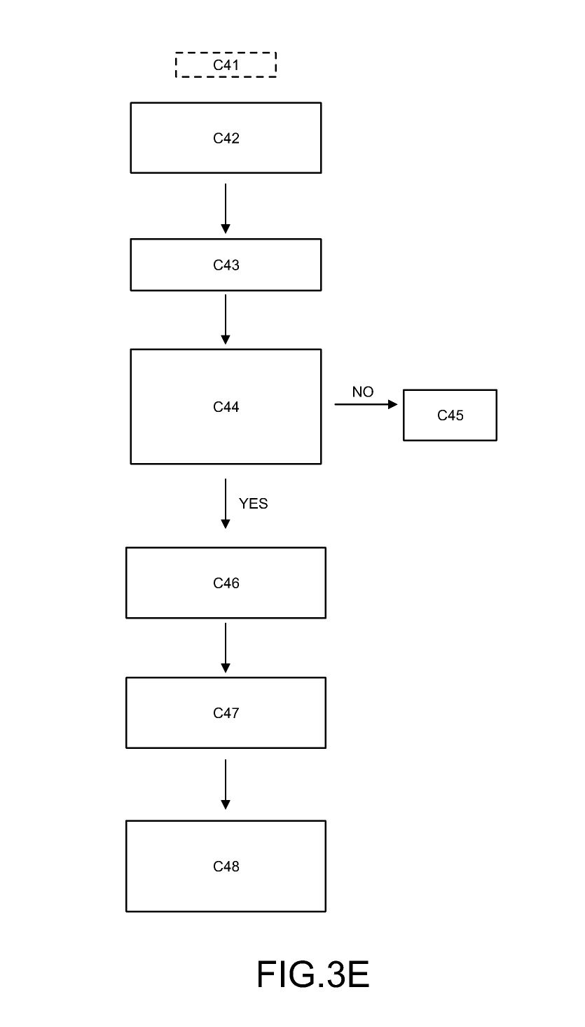

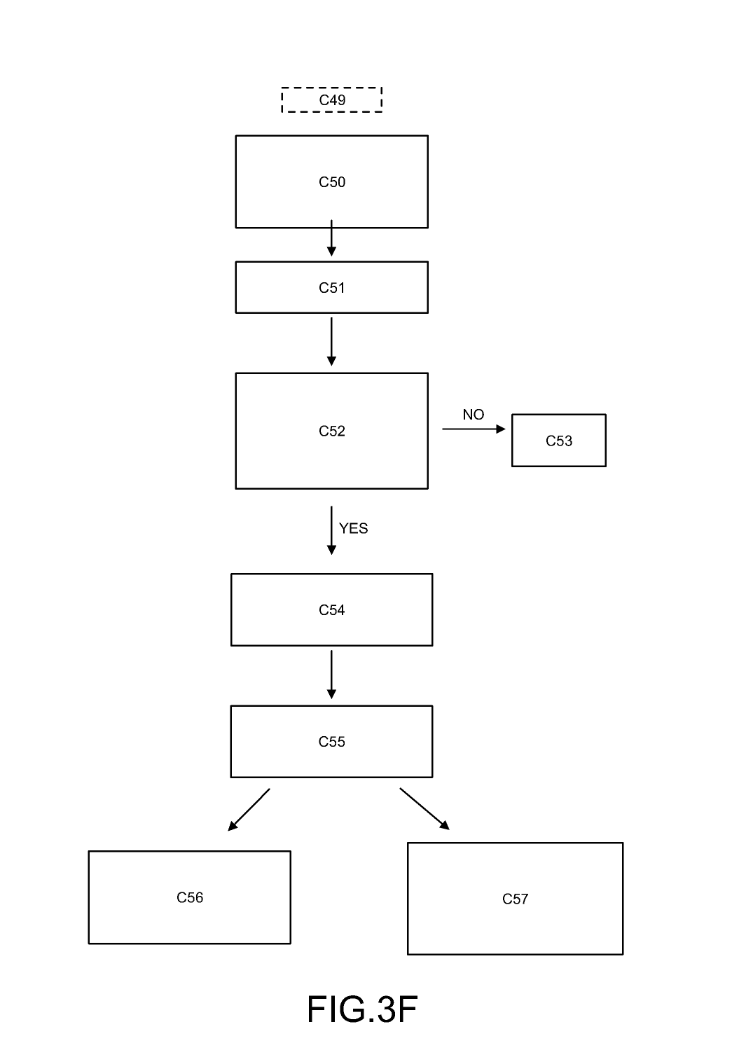

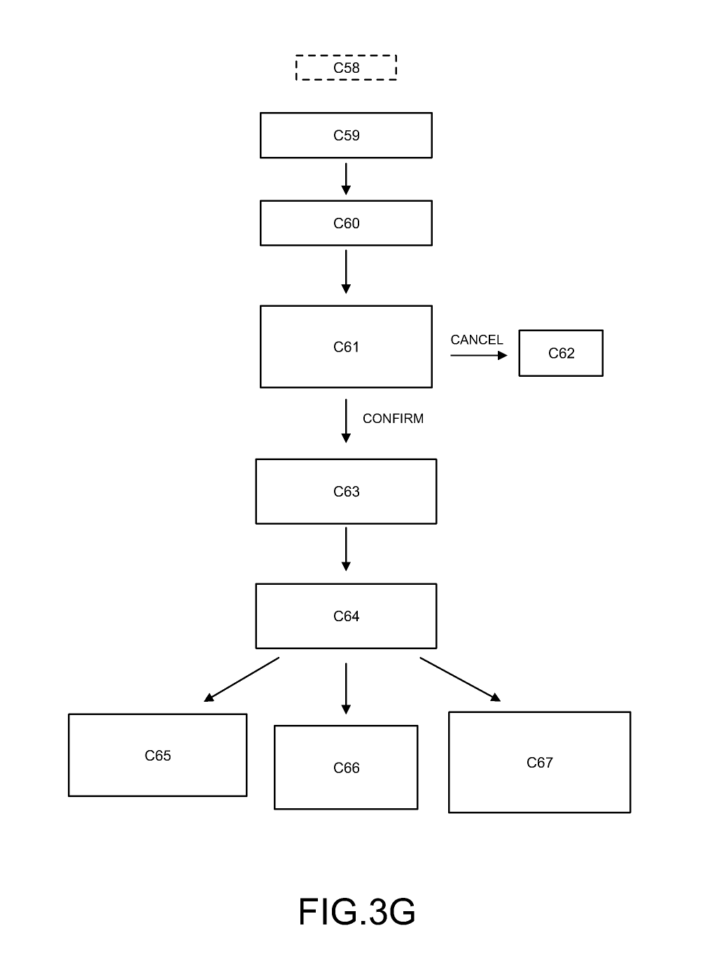

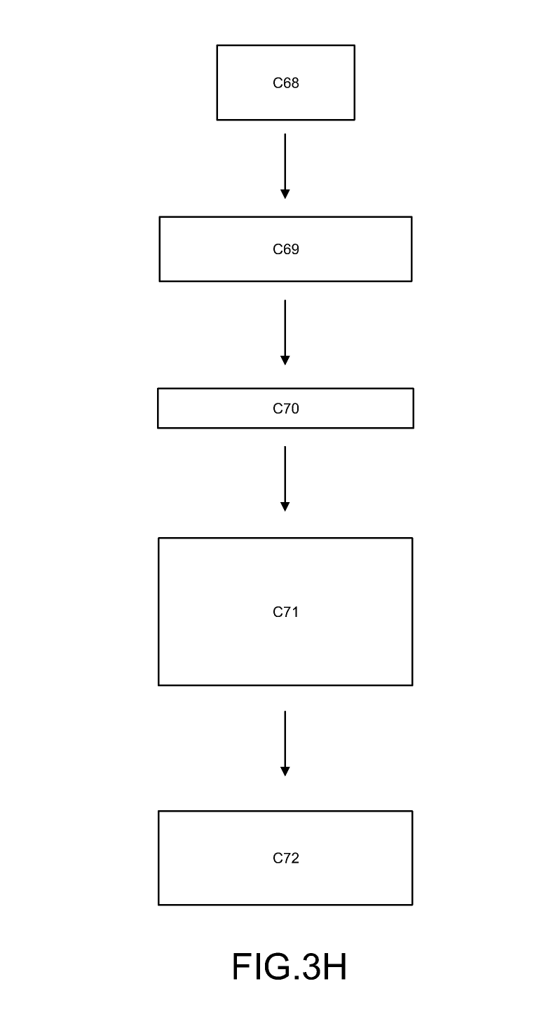

FIG. 3 illustrates a general flowchart relative to access to the on-duty system from professional (Coordinator User);

FIG. 3A illustrates a specific flowchart of one of the many modules foreseen in the flowchart presented in FIG. 3, being the present flowchart relative to the Module—“Doctors”;

FIG. 3B illustrates a specific flowchart of another of many modules foreseen in the flowchart presented in FIG. 3, being the referred flowchart relative to the Module—“Scale Set”;

FIG. 3C illustrates a specific flowchart of another of many modules foreseen in the flowchart presented in FIG. 3, being the mentioned flowchart relative to the Module—“Scale Adm.”;

FIG. 3D illustrates a specific flowchart of another of many modules foreseen in the flowchart presented in FIG. 3, being the referred flowchart relative to the Module—“Scale”;

FIG. 3E illustrates a specific flowchart of one of the Interactive Actions in Scale—To Get On-duty, flowchart which is derived from flowchart in FIG. 3D;

FIG. 3F illustrates a specific flowchart of another of the Interactive Actions in Scale—To Make Available On-duty;

FIG. 3G illustrates a specific flowchart of another of the Interactive Actions in Scale—Request Changes;

FIG. 3H illustrates a specific flowchart of one of the many modules foreseen in the flowchart presented in FIG. 3, being the present flowchart relative to the Module—“On-duties”;

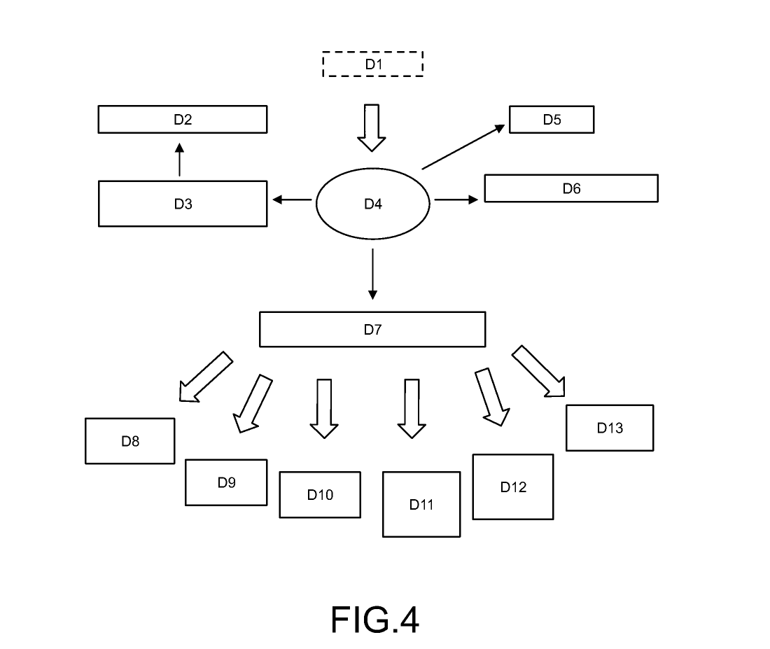

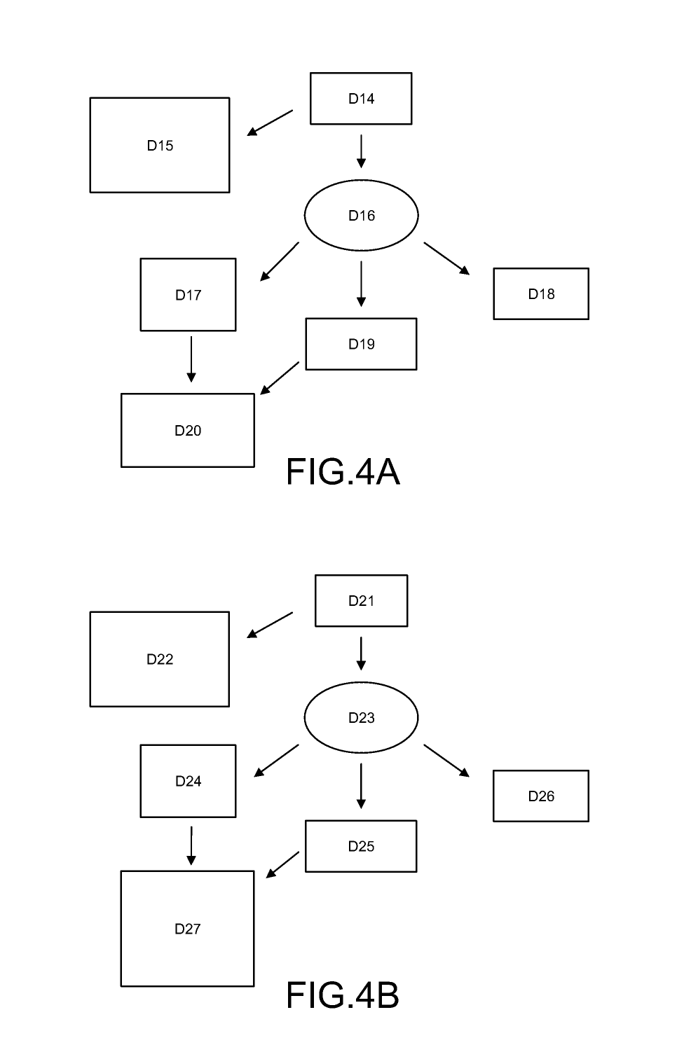

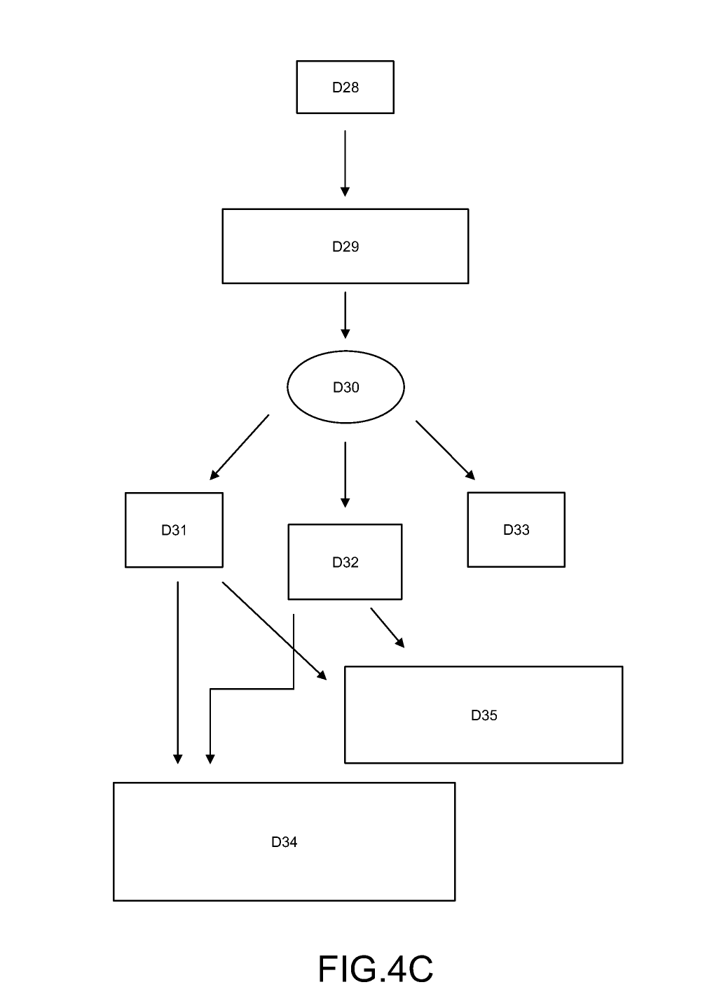

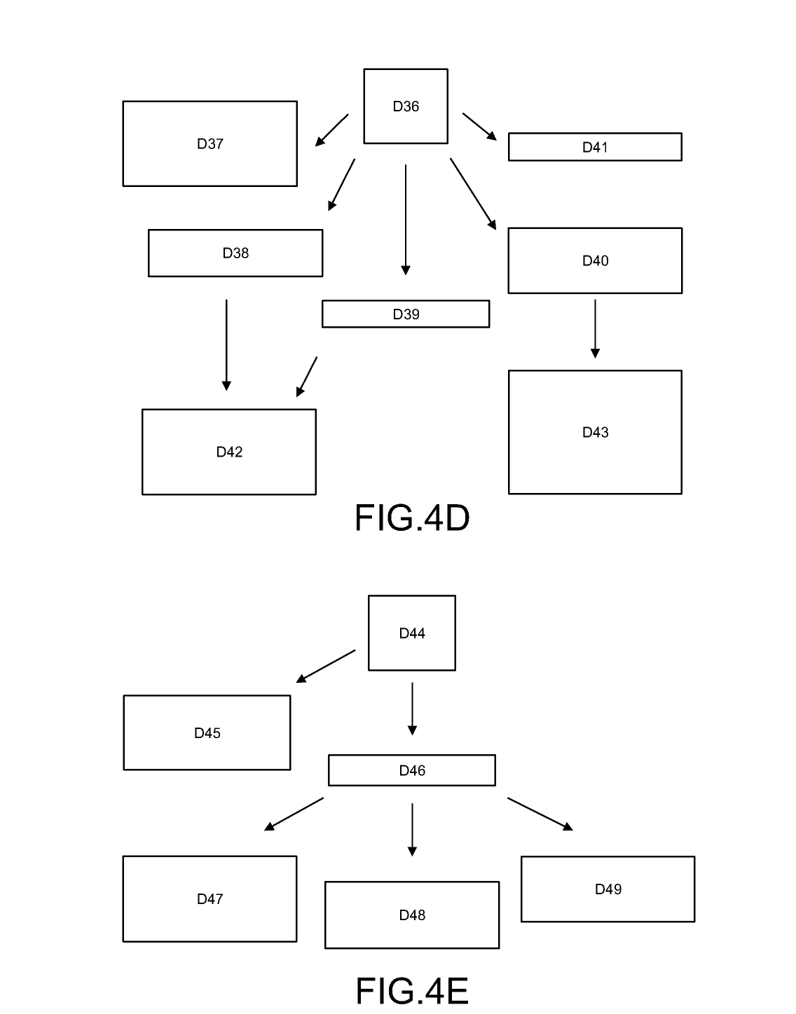

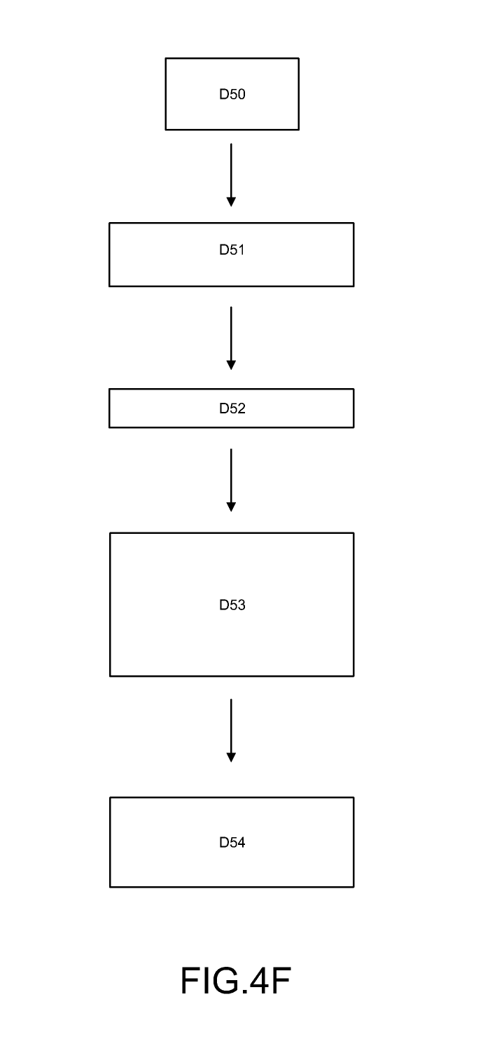

FIG. 4 illustrates a general flowchart relative to access to the on-duty system from professional (Administrator User);

FIG. 4A illustrates a specific flowchart of one of the many modules foreseen in the flowchart presented in FIG. 4, being the present flowchart relative to the Module “Services”;

FIG. 4B illustrates a specific flowchart of one of the many modules foreseen in the flowchart presented in FIG. 4, being the referred flowchart relative to the “Module Staffs”;

FIG. 4C illustrates a specific flowchart of another of many modules foreseen in the flowchart presented in FIG. 4, being the present flowchart relative to the Module “Doctors”;

FIG. 4D illustrates another specific flowchart of another of many modules foreseen in the flowchart presented in FIG. 4, being the present flowchart relative to the “Module Scale Set”;

FIG. 4E illustrates another specific flowchart of another of many modules foreseen in the flowchart of FIG. 4, being the present flowchart relative to the “Module Scale Adm.”;

FIG. 4F illustrates more a specific flowchart of another of many modules foreseen in the flowchart presented in FIG. 4, being the present flowchart relative to the Module “On-duties”; and

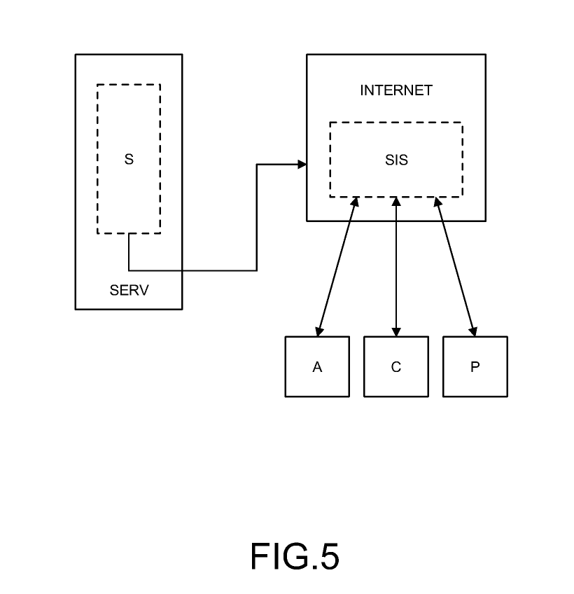

FIG. 5 illustrates a schematic diagram of the equipment employed for implementing the system here proposed, being further retreated the connection of this equipment with the internet environment, where the system for managing and coordinating health area staffs that work under on-duty regime may be accessed.

According to how much the figures related above are illustrated, the object process of this invention patent application establishes the implantation of a system that offers an innovative solution for these difficulties, allowing the creation and maintenance of an official online scale, available 24 hours per day and that may be accessed by all member of a certain staff.

It is an online platform, of interactive scales and that provides a system that through a web platform, allows the interaction between coordinators and on-duty doctors, with an online on-duty scale always updated, and that offers the possibility of interactive actions as, for example, taking over an available on-duty straight through the site, without the necessity of getting in touch with the responsible for staff administration.

In this concept, there would be a time saving of the on-duty doctors and coordinators, smaller chances of mistakes in the scale organization and a bigger agility in the information exchange between the members of medical staffs.

The system described herein predicts the use of a software to act as facilitator in the interaction between health professionals, medical staffs and hospital services managers. All this through an online platform of interactive scales.

The conventional users will have the ability to view their appointments with the staff in which they work through the web or mobile devices or any other internet access form. They will have at their disposal a great number of interactive resources as the option to application time changes, taking over available on-duty, making a certain period available for another colleague or exchange messages with another users or coordinators. All restricted to their respective staff.

The system described herein presents a series of advantages, which may be listed:
The creation of an online official scale available 24 hours per day, always updated;
Scale interactivity, allowing on-duty doctors to perform actions such as taking over on-duty or exchanging on-duty through some few steps;
Possibility of editing and administering the scale by the coordinators, changing with total freedom the number of on-duty of a certain day, or the on-duty specific assigned doctors;
Communication through e-mail and SMS to the coordinators of each action performed in its staff;
Easy interaction between on-duty doctors and coordinators;
Smaller risk of on-duty absence or scales errors;

The coordinators may register on-duty doctors in its staffs, edit on-duty on-duties scales, release scales for viewing and interaction of conventional users, exchanging messages with other users, write notices to its staff, or also, excluding doctors of its staff.

Thus, the present system further presents the following advantages:
Access to an exclusive system to registered users in each staff;
Control of staffs by the clinical board, through a simple access to the system;
Detailed spreadsheet of on-duty performed by each user, in a certain period, making it easy for on-duty accounting;
Auditing system of platform, with the possibility of obtaining information about which user performed each action, when, and through which IP address.

All functions are restricted to its respective staff and with their coordinators' authorization.

Each one of the actions performed by each on-duty doctor is informed to the coordinators of its respective staff through e-mail or SMS.

The system described herein further presents optional functions, which are:
Notices via SMS about on-duties for users, and about actions performed in the system for coordinators;
Interface with message exchange between users;
Apps for IOS, Blackberry and Android, with PUSH alerts;
Possibility of integration with a biometric reading system in the health services, for registering the moment of entrance and exit of each professional;
Platform with possibility of extension for other health professionals, such as nurses, nursing technicians, physiotherapists, speech therapists, etc.;
Possibility of specific adjustments for solving questions related to a certain health service;

The system described herein represents an innovation regarding to health administration sector, innovation that is translated as:

a) Defining health services such as clients, and not on-duty doctors, which will only be users without a system utilization cost.

b) Offering complete independence to the coordinator, respecting the medical staff integrity, i.e., doctors non-authorized by the coordinator do not view the scale or interact with its staff.

c) Allowing the user to interact with the service to view, accept, change or make on-duty available without the necessity of direct communication with the coordination staff, and at any time.

Regarding to the drawings illustrating this invention patent application, FIG. 1 that illustrates a general flowchart relative to the interaction between the health service and the many staffs of management and coordination and its respective professional boards.

In this flowchart, it is represented: the Health Service (A1), that may have one more medical staffs that operate in on-duty scale, divided according to its specialty or place of acting. E.g., ER Medical Clinic staff; ER Pediatrics Staff; Orthopedics Staff; Gynecology and Obstetrics Staff; ICU Staff; etc.

Another block represented in FIG. 1 is the one of many Staffs: Staff A; Staff B and Staff C, respectively indicated as (A2), (A3) and (A4), being that each one of the staff is managed by one or more coordinators and that contain many on-duty doctors, being that the coordinators people are indicated by C circles.

The C coordinators are responsible for keeping the integrity of the scale of its respective staff, organizing and distributing the on-duty doctors or on-duties.

The same FIG. 1 further includes the figure of the on-duty doctors (P), which are doctors that constitute each one of the staffs of the respective health service. The questions that need to be solved such as on-duty change, taking over on-duties or passing on-duty are reported to the coordinator(s) of its staff.

FIG. 2 illustrates a general flowchart relative to access the on-duty system from professionals (On-Duty Users).

The access to the system by the on-duty doctor user (block B1) requires a valid login and password.

The same FIG. 2 further includes the block of Action Options (B4), to which are related, by one side, the block “Access to Personal Settings” (B3), which is related to the block “Change access password” (B2); and by another side are related to the block “Logout” (B5) and “Choose new staff (B6). Regarding to the “Choose new staff” (B6) block, it shall be noted that each user may be integrating one or more staffs, of the same service or distinct services.

The “Action Options” block (B4) derives to the block “Select Module” (B7) and from this to the block “Scale Module” (B8), which is the only available module for the On-Duty Users. The “Module: Scale” (B8) block derives to the block “Scale View of its respective Staff” (B9), which corresponds to the condition that is always presented to the selected staff scale in its last access, in case users integrate one or more staffs.

The block relative to the “Module: Scale” (B8) derives to the “Action Options” block (B11) and from this to the block “Actions in the change window: answering or canceling an application” (B12), or further to the block “Interactive Actions in Scale:—To Get On-duty,—To Make Available On-duty,—Requesting changes (B13).

The same “Action Options” block (B11) derives to the block “Choose new month and year to view the Scale” (B10). By “default”, the system access always presents scales of the current month.

FIG. 2A illustrates a specific flowchart to one of the “Interactive Actions in Scale” (B13)—“To Get On-duty”, flowchart that derives from the flowchart presented in FIG. 2.

Quoted FIG. 2A presents the block “To Get On-duty” (B14), and next the block “Identifying vacant or available on-duty” (B15), being that vacant or available on-duty are marked in red in scale. The on-duty doctor interested may take over a certain on-duty clicking straight on it in the scale screen.

The block (B15) derives to the block “Click on the chosen on-duty” (B16), and from this part to the block “Message: “Do you wish to choose on-duty from day 21/11/13 in the period 19:00-07:00? You will be responsible for this time!” (B17). In negative case, starts the block “Operation Cancelled” (B18) and in affirmative case starts the block “Message: “On-duty chosen successfully!” (B19).

From the block B19 starts the block “Name of the allocated user in the chosen time in Scale” (B20) and from this to the block “Coordinator(s) has(have) received an e-mail communicating the action performed by the on-duty doctor” (B21).

FIG. 2B also illustrates an specific flowchart to one of the Interactive Actions in Scale (B13) relative to “Making Available On-duty” (B22), flowchart which also derives from the flowchart presented in FIG. 2.

In FIG. 2B is foreseen the block “Identifying the user on-duty responsibility” B23), being that the user has its on-duty highlighted in blue.

From the block (B23) starts the block “Click on the chosen on-duty” (B24) and from this starts the block “Message: “Do you wish to make available on-duty from day . . . in the period . . . ? until someone chooses this on-duty, you will still be responsible for it!” (B25), being that in negative case, starts the block “Operation Cancelled” (B26), and in affirmative case, starts the block “Message: “On-duty made available successfully!” (B27).

In the sequel to the block (B27), starts the block “The on-duty made available is highlighted in red” (B28), being that from now on, other users can take over the referred available on-duty. The availability may be cancelled in case on-duty has not yet been taken over.

From the block (B28) starts the block “Coordinator(s) has(have) received e-mail communicating the action performed by the on-duty doctor” (B29) and further to the block “If the on-duty is available, the user can cancel the availability by clicking on the referred on-duty.” (B30).

FIG. 2C also illustrates a specific flowchart to another of the Interactive Actions in Scale (B13) that is the “Application Changes” (B31), flowchart that is equally derived from the flowchart presented in FIG. 2;

The mentioned FIG. 2C includes the block “Identifying the wanted on-duty) (B32), being that it is not possible to application change in case the wanted on-duty is already scaled to the requesting on-duty doctor or there is a vacant on-duty.

From block (B32) starts the block “Click on the chosen on-duty” (B33) and from this starts the block “Appears window with user on-duties that may be offered in exchange” (B34), being that in case of cancelling, starts the block “Operation Cancelled” (B35) and in case of confirmation starts the block “Message: ‘Application performed successfully!’” (B36).

From the block (B36) starts the block “On-duty involved become highlighted in green” (B37), from which derives block “Coordinator(s) has(have) received e-mail communicating the action performed by the on-duty doctor” (B38); “The target on-duty doctor of the application received a notice by e-mail” (B39); and “If the change has not yet happened, the requesting user can cancel the application in the exchange window” (B40).

FIG. 3 illustrates a general flowchart relative to access the on-duty system from professional (Coordinator User) (C1), being that for this case it is necessary “Valid Login and Password”.

The quoted FIG. 3 predicts the “Action Options” block (C4), from which derives, by one side the block “Access to Personal Settings” (C3) and by other side to the block “Logout” (C5) and also to the block “Choose new staff” (C6), being that each user may be integrating one or more staffs, from the same service or from different services.

From the block “Access to Personal Settings” (C3) starts the block “Change access password” (C2).

Further from the “Action Options” block (C4) starts the block “Select Module” (C7) and from this starts the block “Module: Doctors” (C8) that presents the list of doctors registered in the respective staff, in alphabetical order, making possible to include, add or exclude the staff doctors; “Module: Scale Set” (C9), which allows the scale customization with definition of on-duty in each day of the week, with its start and end times, and amount of scaled professionals; “Scale Module Adm.” (C10), which presents scale for editing specific dates, allowing the time change and the amount of on-duty, of the scaled professionals and other options; “Scale Module” (C11); and the module “On-duty” (C12), that make possible to research on-duty performed in a certain period of time determined by a specific professional. There is the option to export the result to an “.xls” file. It further allows to associate values to on-duty, with calculating the amount to be received by the concerned professional.

FIG. 3A illustrates a specific flowchart of one of the many modules foreseen in the flowchart presented in FIG. 3, being the present flowchart relative to Module—“Doctors (C13)”, which presents the list of the doctors registered in the respective staff, in alphabetical order. It makes possible to include, to edit or to exclude the staff doctors.

From the block “Module Doctors” (C13) starts the block “Present the list of the doctors registered in the respective staff” (C14) and from this starts the “Action Options” block (C15).

From the block “Action Options (C15) starts the block “Insert New Doctor” (C16); to the block “Edit Registered Doctor” (C17); and to the block “Exclude Doctor from the Staff” (C18), being that the doctor is excluded from the staff concerned, but keeps access to system through his login and password, being able to act in other staffs in which is normally registered.

From the block “Insert New Doctor” (C16) starts the block “Data: CPF; CRM; Name; Name for Viewing in Scale; Sex; Specialty; E-mail; Date of birth; Address; State; City; Phone 1; Phone 2; Undergraduate University” (C20); and to the block “—Option to create user to the doctor, in case it is not yet existent.—Option to make the coordinator-doctor of the respective staff.” (C19).

From the block “Edit Registered Doctor (C17) steps also to the block “Data: CPF; CRM; Name; Name for Viewing in Scale; Sex; Specialty; E-mail; Date of birth; Address; State; City; Phone 1; Phone 2; Undergraduate University” (C20), already quoted before; or to the block “—Option to create user to the doctor, in case it is not yet existent.—Option to make the coordinator-doctor of the respective staff.” (C19).

FIG. 3B illustrates the specific flowchart of another of the many modules foreseen in the flowchart presented in FIG. 3, being the referred flowchart relative to Module—“Scale Set” (C21), which allows customization of the scale with definition of the on-duties in each day of the week, with start and end times, and amount of scaled professionals. There is yet the option to choose the scaled professionals for each day of the week, setting that will repeat in scale and may be changed in specific dates in the “Scale Module Adm.” (C10).

From the block relative to “Scale Module Set” (C21) starts the block “Determine periodicity of settings: Weekly or Fortnightly” (C22); “Create new On-duty” (C23); “Edit Existing On-duty” (C24); “Edit settings of a specific day of the week” (C26); and “Exclude Existing On-duty” (C25).

From the block “Create new On-duty” (C23) starts the block “Date: Start time; End time; Amount of On-Duty Doctors; Note” (C27), block from which starts also the “Edit Existing On-duty” (C24) block.

From the block “Edit settings of a specific day of the week” (C26) starts the block “Create, Edit or Exclude On-duties; Choose the scaled professionals for each On-duty0; Add or Edit note” (C28).

FIG. 3C illustrates a specific flowchart of another of the many modules foreseen in the flowchart presented in FIG. 3, being the mentioned flowchart relative to Module—“Scale Adm.” (C29), the “Scale Module Adm.” presents scale for editing of specific dates, allowing the change of the times and amounts of on-duties, of the scaled professionals and other options.

From the block “Scale Module Adm.” (C29) starts the block “Choose new month and year to view the Scale” (C30) and yet to the block “Edit specific day” (C31).

Regarding block “Choose new month and year to view the Scale” (C30), by “default”, the access to system always presents the scale of the current month. The coordinator visualizes past and future dates through this module.

From the block “Edit specific day” (C31) starts the block “Create, Edit or Exclude On-duties; Add or edit note.” (C32); to the block “Choose the scaled professionals for each on-duty” (C33); and to the block “Releasing Scale for On-Duty Doctors Viewing” (C34).

FIG. 3D illustrates a specific flowchart of another of the many modules foreseen in the flowchart presented in FIG. 3, being the referred flowchart relative to Module—“Scale” (C35).

The quoted 3D Fig. presents the block “Action options” (C38); From which derive the blocks “Actions in the exchange windows: answering or cancelling applications” (C39); “Interactive actions in Scale:—Get on-duties—Making on-duties available, and Requesting changes” (C40) and yet the block “Choose new month and year to view the Scale” (C39).

Still regarding block “Module: Scale” (C35) we know that the same derives from the block “Viewing of the Scale of its respective staff” (C36).

FIG. 3E illustrates a specific flowchart of one of the Interactive Actions in Scale—Get On-duties (C41), flowchart which is derived from flowchart of the FIG. 3D.

In FIG. 3E quoted the block “Identifying the vacant or available on-duties” (C42) is foreseen, where the vacant or available on-duties are marked in red in the scale. The on-duty doctor interested can take over a determined on-duty clicking on it straight in the scale screen.

From the block “Identifying the vacant or available on-duties” (C42) starts the block “Click on the chosen on-duty” (C43) and from this, the block “Message: “Do you wish to choose on-duty from day 21/11/13 in the period of 19:00-07:00? You will be the one responsible for this time” (C44), being that, in negative case, starts the block “Operation Cancelled” (C45) and in affirmative case, starts the block “Message: “On-duty chosen successfully!” (C46).

From the block “Message: “On-duty chosen successfully!” (C46) starts the block “Name of the allocated user in the chosen time in Scale” (C47) and from this block starts the block “Coordinator(s) has(have) received e-mail communicating the action performed by the on-duty doctor” (C48).

FIG. 3F illustrates a specific flowchart of another of the Interactive Actions in Scale Making On-duties Available (C49);

In the quoted FIG. 3F) the block “Identifying on-duties of user's responsibility, vacant or available” (C50) is foreseen, where the user has its on-duties highlighted in blue, from which starts the block “Click on the chosen on-duty” (C51) and from this to the block “Message: “Do you wish to make available the on-duty of the day . . . in the period . . . ? Until someone chooses this on-duty, you will still be responsible for it!” (C52) being that in negative case, starts the block “Operation Cancelled” (C53) and in affirmative case, starts the block “Message: “On-duty made available successfully!” (C54), being that, next, “The on-duty made available becomes highlighted in red” (C55), and from this block starts the block “Coordinator(s) has(have) received e-mail communicating the action performed by the on-duty doctor” (C56); and “If the on-duty is available, the user can cancel its availability by clicking on the referred on-duty” (C57).

FIG. 3G illustrates a specific flowchart of another of the Interactive Actions in Scale—and part of this block “Application Changes” (C58); flowchart which is also derived from the flowchart of the FIG. 3D.

In the quoted FIG. 3G the block “Identifying the desired on-duty” (C59) is foreseen, from which starts the block “Click on the chosen on-duty” (C60) and from this “Appear window with the user's on-duties that may be offered in exchange” (C61). In negative case, starts the block “Operation Cancelled” (C62) and in affirmative case, starts the block “Message: “Application performed successfully!” (C63), being that, next, “The on-duties involved become highlighted in green” (C64) and, from this come the blocks: “Coordinator(s) has(have) received email communicating the action performed by the on-duty doctor (C65); “The on-duty doctor target of the application received notice by email” (C66); and optionally “If the change has not yet happened, the requesting user can cancel the application in the window of changes” (C67).

FIG. 3H illustrates a specific flowchart of one of the many modules foreseen in the flowchart presented in FIG. 3, being the present flowchart relative to Module—“On-duties” (C68); from which follows blocks: “Define Start Date, Final Date and the Professional.” (C69); “Perform Research” (C70); “Viewing the on-duties performed, in the interval of time determined, by the chosen professional” (C71); and yet there is an “Option to generate spreadsheet in the .xls format with the research result” (C72).

FIG. 4 illustrates a general flowchart relative to access to on-duty system from professional (Administrator User) indicated by block (D1).

The FIG. 4 mentioned, from which block (D1) starts the “Action Options” block (D4), from which derives the block “Access to Personal Settings” (D3); “Logout” (D5); “Choose new staff” (D6), being that each user may be integrating one or more staffs, from the same service or different services; and block “Select Module (D7).

From the block “Access to Personal Settings” (D3) starts the block “Change access password” (D2).

From the block “Select Module” (D7) starts the blocks: “Module: Services” (D8), which allows the viewing of the services registered in the system, to include new services, and to edit, or to exclude services registered; “Module Staffs” (D9), that allow viewing of the staffs registered in the system, with its respective specialties. It also makes possible to include new staffs, and to edit or to exclude staffs; “Module Doctors” (D10), that present a list of the doctors registered in the respective staff, in alphabetical order. It makes possible to include, to edit or to exclude staff doctors; “Scale Module Set (D11), that allows customization of the scale with definition of the on-duties in each day of the week, with start and end times, and amount of scaled professionals. There is the option to choose the scaled professionals for each day of the week, setting which will be repeated in the scale and may be changed in specific dates in the Module Scale Adm.; “Scale Module Adm.” (D12), that presents the scale for edition of specific dates, allowing time change and amounts of on-duties, of the scaled professionals and other options; and “Module on-duties” (D13), that makes possible to research the on-duties performed and a period of time determined by a specific professional. There is the option to export the result to an “.xls” file. It allows associating values to on-duties, with a calculus of the value to be received by the concerned professional.

FIG. 4A illustrates a specific flowchart of one of the many modules foreseen in the flowchart presented in FIG. 4, being the present flowchart relative to the Module “Services (3)” (block D14), that allow viewing the services registered in the system, including new services, and editing or to excluding the services registered.

From the block (D14) derive the blocks “Present the list of services registered in the System” (D15) and the “Action Options” block (D16), being that from the quoted block (D16) start blocks “Insert New Service” (D17); “Edit Service” (D19); and “Exclude Service” (D18). The blocks (D17) and (D19) derive to the block “Date: Name and Name for Viewing” (D20).

FIG. 4B illustrates a specific flowchart of another of the many modules foreseen in the flowchart presented in FIG. 4, being the present flowchart relative to “Module Staffs” (D21).

The block “Module Staffs” (D21) goes to block “Present the list of the Staffs registered in the System (D22) and to the “Action Options” block (D23), being that the block (D23) goes to the blocks “Insert New Staff” (D24); “Edit Staff” (D25); and “Exclude Staff” (D26). The blocks (D24) and (D24) and (D25) go to block “Date: Name; Name for Viewing; Service and Specialty” (D27).

FIG. 4C illustrates another specific flowchart of another module foreseen in the flowchart portrayed in FIG. 4, being the quoted flowchart of FIG. 4C relative to “Module Doctors” (D28), which presents the list of the doctors registered in the respective staff, in alphabetical order, making it possible still to include, edit or exclude the staff doctors.

In the quoted FIG. 4C the block (D28), go to the block “Present the list of the doctors registered in the respective staff” block (D29), which goes to the “Action Options” block (D30).

Block (D30) goes to blocks: “Insert New Doctor” (D31); “Edit Registered Doctor” block (D32); and “Exclude Staff Doctor” (D33).

Block (D31) goes to blocks “Date: CPF; CRM; Name; Name for Viewing in Scale; Sex; Specialty; E-mail; Date of Birth; Address; State; City; Phone 1; Phone 2; Undergraduate University” Block (D34); and “—Option to create user to the doctor, in case it is not yet existent.—Option to become the coordinator-doctor of the respective staff.” Block (D35). For the quoted block (D35), converge also the block “Edit Registered Doctor (D32) and the block “Insert New Doctor” (D31).

FIG. 4D illustrates another specific flowchart of another of the many modules foreseen in the flowchart presented in FIG. 4, being the present flowchart relative to the “Scale Module Set” block (D36).

In FIG. 4D, the block “Module: Scale Set” (D36), that allows customization of the scale with definition of the on-duties in each day of the week, with start and end times, and amount of scaled professionals, goes to blocks: “Determine periodicity of the Settings: Weekly or Fortnightly” block (D37); “Create new On-duty” block (D38); “Edit existent on-duty” block (D39); “Edit Settings of a specific day of the week” block (D40); and “Exclude Existing On-duty” block (D41).

The blocks (D38) and (D39) go to block “Date: Start time; End time; Amount of On-Duty Doctors; Note” (D42). Block (D40) goes to block “Create, Edit or Exclude On-duties: Choose the scaled professionals for each On-duty; Add or edit note.” (D43).

FIG. 4E illustrates a more specific flowchart of another of the many modules foreseen in the flowchart presented in FIG. 4, being the referred flowchart relative to “Module “Scale Adm.” block (D44).

In the quoted FIG. 4E the block (D44) goes to blocks: “Choose new month and year to view the Scale” Block D45); and “Edit specific day” (D46). Block (D46) of FIG. 4E goes to: “Create, Edit or Exclude On-duties; Add or edit note.” Block (D47); “Choose the scaled professionals for each On-duty” Block (D48); and “Releasing Scale for On-Duty Doctors Visualization” block (D49).

FIG. 4F illustrates more a specific flowchart of another of the many modules foreseen in the flowchart presented in FIG. 4, being the present flowchart relative to Module “On-duties” block (D50), that makes possible the research of the on-duties performed in a period of time determined by a specific professional. There is the option of exporting the result to an “.xls” file. It allows the association of values to on-duties, with calculus of the value to be received by the concerned professional.

From “Module On-duties” (D50), starts block “Define Start Date, Final Date and Professional” (D51); from which starts block “Perform Research” (D52), of the referred block (D52) starts the block “Viewing the on-duties performed, in the interval of time determined by the chosen professional” (D53).

From block (D53) goes to block “Option to generate spreadsheet in the .xls format” with the result of the research” (block D54).

FIG. 5 illustrates on its turn, in an objective form, the equipment employed to implement the system here treated, equipment that is represented, schematically, such as a computer which acts as a server (SERV), in which is installed the software (S).

The configured equipment such as a computer that acts as a server (SERV), is connected to internet, that constitutes the virtual environment where the concerned system, indicated by the reference (SIS) may be accessed, under the many conditions previously reported, be it by Administrator (A), such as also by coordinator(s) (C) and still by health area professionals (preferably doctors) (P).

The mentioned FIG. 5 demonstrates the interaction between agents (A), (C) and (P) with the system (SIS) in the internet environment, interaction which makes possible the practical use of the system here treated.

The content of FIG. 5 does not exclude the possibility that the System (SIS) here treated is, for example, stored in a virtual environment such as happens in the cloud systems.

The system here proposed values the respect for the integrity of the health services professional staff. The service is offered to the responsible of each staff, and this professional is who determines which on-duty doctors will be part of its staff. Just the registered users in the respective staff will have access to the interactive scale of on-duties.

The system here proposed is not about a “free market” of on-duties, through which any user has access to on-duties available. An important difference of the treated solution in the present system is the respect for the integrity of the health services professional staff. The service is offered to the responsible for each staff, and this professional is who determines which on-duty doctors will be part of its staff. Just the registered users in the respective staff will have access to the interactive scale of on-duties.

This way, we can list the advantages compared to the competition:
Respect for the staff integrity, offering total independence for the coordinator to manage its own scale and its on-duty doctors.
Possibility of the user to conclude in some seconds the choice of a vacant on-duty, and confirm this decision, accessing his scale at any time of the day. This system dismisses the need of contact between the on-duty doctor and its coordinator for on-duty change to be performed.
Exclusive product offered straight to the health services and to companies administering medical staff.
It is hard for malicious users or fake professionals to act for it depends on the coordinator of the respective staff permission.

In this system, users are divided in three groups of distinct accesses, the conventional users (on-duty doctors), staff coordinators, and the system administrator. The conventional users will be able to see their appointments, with the staff in which works in, via web through mobile devices or any other form of internet access. It will have at its disposal a great number of interactive resources, such as options to application time changes to other users, taking over available on-duties, to make available a determined period to another colleague, or exchanging messages with other users or coordinators. All functions restricted to its respective staff.

The coordinator users will be able to register on-duty doctors in its staffs (with the creation of a login and access password for each one of them), edit on-duty scales, release scales for visualization and interaction of the conventional users, exchanging messages with other users, write notices to its staff, or also, excluding staff doctors.

The system administrator has power to register health services, staffs, conventional users and coordinators. He can also view the users and staffs of the system, as well as editing its data.

Description of the Functionalities of the Present System:

1. Staffs Registry (Agent: Administrator)

The administrator can register a new health service or a new staff in determined health service, view the list of registered staffs and edit data of some staff. The setting of its scale is function of the coordinator of the respective staff (see Functionality 4).

2. Doctors Registry (Agent: Coordinator)

The coordinator can insert a new doctor in its staff, view the list of the doctors registered, edit data of some doctor, and create a user for that doctor (see Functionality 3), or remove him from its staff.

When starting the filling of fields to insert a new doctor, the system verifies by the CPF or by the CRM, if he is already registered in another health service, and exhibits the remaining fields in case he already exists.

Doctor Registry Fields:
CRM [Regional Medical Counsel] (Number and State)
CPF [Individual Taxpayer's ID Number]
Name
Specialty
Email
Address
Phones
Date of Birth
Undergraduate University (optional)

3. Creation of User (Agent: Coordinator)

When inserting a doctor, the system asks to the coordinator if he desires to create a user for that doctor. It is also possible, in the doctor-listing screen, to choose a doctor that still has not a user and to application the creation of a user.

When the coordinator defines the creation of a user to the doctor, the system defines the login as the doctor's email and creates a random password. These data are sent to the doctor by email.

4. Scale Setting (Agent: Coordinator)

In this functionality the coordinator can create (or edit in case already existent) the scale settings of the staff in which is registered.

Presents the following possibilities:

Defines weekly or fortnightly periodicity. When changing this value, a grid of 7 or 14 days will be shown.

Defines the possible periods of on-duties to scale (Ex.: 07 AM-07 PM, 07 PM-07 AM).

Defines a standard amount of needed professionals by on-duty (Ex.: 04).

To a determined day, define the amount of needed professionals (e.g. On Thursdays, 03 professionals).

Allocate a professional by standard in determined on-duty (e.g. Dr. Danielle every Tuesday, from 07 AM to 07 PM).

5. Scale Administration (Agent: Coordinator)

By accessing the scale creation screen, the user inserts a start and end date (by default the current month). The grid with already existent on-duties is exhibited, released or not for viewing and interaction of the conventional users. For days still not planned, on-duties will be exhibited according to a setting of the scale.

For each period of one day, there is the possibility of changing the time of the period, changing the amount of needed professionals, and allocating professionals.

There will also be the option of releasing the selected period for access of the conventional users, allowing that these visualize and interact with the released period of the scale.

6. Access to Scale (Agent: Conventional)

When accessing the Scale View screen, the user can choose a month to be exhibited (by default the current month). The conventional user can only see the future dates that are released by the coordinator of its staff.

Possibilities of User Interaction:

The user can choose a vacant time to register, with a confirmation message coming up, remembering the user that the time responsibility will be his from that moment on, if he confirms his intention of performing that on-duty. Having the user confirmation, his name completes the available time of the scale, and an e-mail is sent to the staff coordinators, informing them.

One of his times may be made available. A message communicating the user that from that moment on, his times is available to other, but until someone registers, the responsibility is still from the current user. An e-mail is sent to the staff coordinators informing them about the available on-duty.

The user can choose a time of another on-duty doctor to application change. An e-mail is sent to the responsible of the time to be changed, with a link to an area where he can confirm the change or refuse it. The response generates a return e-mail to the doctor who has made the application, informing him if it was accepted or not. In case the change has been performed, it also generates an e-mail to staff coordinators.

7. Consult on-Duties Performed by the Doctor (Agent: Coordinator)

The coordinator inserts a period and chooses a professional. A list of on-duties performed in the staff and a total amount is exhibited. On-duties can only be exported to a CSV file (recognized by Excel).

Technological Characteristics:

The technologies used to the software development that makes possible the system here treated is of the most recent available, and enough to the current system requisites. The system is developed in Java Enterprise Edition version 6 (JEE 6) platform. The main JEE platform technologies and frameworks open source used for system programming are:

JSF (Java Server Faces) 2-JEE specification for creation of the visualization layer based in the MVC (Model-View-Control) model and in the use of reusable components. The implementation of specification being used is RI (Reference Implementation) supplied by Oracle itself.

Spring 3—Framework present in all Java applications layers, providing mainly attendance to non-functional requisites. The official documentation is available in the address:

http://static.springsource.org/spring/docs/3.0.x/spring-framework-reference/html. Subproject Spring Security is used to application access control and possesses a documentation in:

http://static.springsource.org/spring-security/site/docs/3.0.x/reference/springsecurity.html.

Hibernate 3.5—Framework that implements the Java Persistence API—(JPA) pattern of JEE, oriented to integration and object-relation mapping between the app and database. Official documentation in the address: http://docs.jboss.org/hibernate/core/3.5/reference/pt-BR/html/.

Database

The database system used is PostgreSQL (version 8.4), that is the most robust open source database. All documentation including the instructions for installation of the database in Linux operational systems may be found in the site http://www.postgresql.org.

Acceptance of PostgreSQL has been opening to beyond the open source community. Among its users are big international companies, government bodies of many countries and worldwide renowned universities.

Application Server

The application server is the system making available the necessary environment for application operations. It complies with the role of receiving HTTP requisitions originated from WEB, treat these requisitions by means of classes of the application which is hosted and returning to the client the HTML answer generated also by the app. The application server used is Apache Tomcat 7 (http://tomcat.apache.org/).

1. System for managing and coordinating health area staffs that work under on-duty regime, wherein it predicts the use of an online computer platform, where data referring to the on-duty availability, such as dates and times may be accessed and requested by the registered professionals to offer this kind of service; the online platform, of interactive scales provide a system that, through a web platform, allows the interaction between coordinators and on-duty doctors, with an updated online on-duty scale, and that offers possibility for interactive actions.

2. System for managing and coordinating health area staffs that work under on-duty regime, according to claim 1, wherein a platform allowing interactive actions may include: taking over an available on-duty straight from the site; application time changes to other users, to make available a determined period to another colleague, or exchanging messages with other users or coordinators; edition and administration of the scale by the coordinators; the platform still allows the communication through e-mail and SMS to coordinators of each action performed in its staff; easiness of interaction between on-duty doctors and coordinators, being that coordinators may register on-duty doctors in its staffs, edit on-duty scales, release scales for visualization and interaction of the conventional users, exchanging messages with other users, write notices to its staff, or also, excluding staff doctors; the present system still allows the access to system exclusive to registered users in each staff; staff control by the clinical board, through system access; detailed spreadsheet of on-duty performed by each user, in a certain period, making it easy to account on-duty; platform auditing system, with possibility of obtaining information about which user performed a determined action, when, and through which IP address, being that each one of the actions performed by each on-duty doctor is informed to coordinators of its respective staff through e-mail or SMS.

3. System for Managing and Coordinating Health Area Staffs that Work Under on-Duty Regime, according to claim 1, wherein the present system predicts that individuals accessing thereof may be divided in three groups of different access which are: the conventional users (on-duty doctors), staffs' coordinators, and system administrator, being that the conventional users can view its appointments with a staff in which they work, via web through mobile devices or any other form of internet access, having at its disposal options to application time changes to other users, taking over on-duties available, to make available a determined period to another colleague, or exchanging messages with other users or coordinators; the coordinator user may register on-duty doctors in its staffs with the creation of login and access password for each one of them, edit on-duty scales, release scales for visualization and interaction of the conventional users, exchanging messages with other users, write notices to its staff, or also, excluding staff doctors; the system administrator has power to register health services, staffs, conventional users and coordinators, and he can also view system users and staffs, as well as edit its data.

4. System for managing and coordinating health area staffs that work in an on-duty regime, according to claim 1, wherein the present system predicts the following guidelines, which covers its functionalities: 1—Staffs Registry (agent: Administrator): the administrator can register a new health service or a new staff in a determined health service, view the list of registered staffs and edit data of some staff. The scale setting is function of the coordinator of the respective staff (see Functionality 4); 2—Doctors Registry (agent: Coordinator): the coordinator can insert a new doctor in its staff, view the list of the doctors registered, edit data of some doctors, create a user for that doctor, or remove him from its staff, being that by start the filling of fields to insert a new doctor, the system verifies personal data from the professional that may include: CPF or CRM, if he is already registered in another health service, and the remaining fields are exhibited in case he already exists: Doctor registry fields: CRM (Number and State); CPF; Name; Specialty; Email; Address; Phones; Date of Birth; Undergraduate University; 3—Creation of User (agent: Coordinator): when inserting a doctor, the system asks the coordinator if he wishes to create a user for that doctor, being also possible, in the doctor listing screen, to choose a doctor that still have no user and to application the creation of a user; when the coordinator defines the creation of a user to the doctor, the system defines the login as the doctor's e-mail and creates a random password, being these data sent to the doctor by e-mail; 4—Scale Setting (agent: Coordinator): the coordinator can create (or edit in case already existent) the scale settings of the staff in which is registered, presenting the following possibilities: a) define a weekly or fortnightly periodicity, being that when changing this value, a grid with 7 or 14 days will be exhibited; b) define the possible periods of the on-duties to scale; c) define a standard amount of needed professionals by on-duty; d) allocate a professional by standard in a determined on-duty; 5—Scale Administration (agent: Coordinator): when accessing the scale creation screen, the user inserts a start and end date (by default the current month), being that the grid with already existent on-duties is exhibited, released or not for viewing and interaction of the conventional users, being that for days still not planned, it will be exhibited the on-duties according to a setting of the scale; for each period of one day, there is the possibility of changing the time of the period, change the amount of needed professionals, and allocate the professionals; there is also the option to release the selected period to access to the conventional users, allowing that this visualize and interact with the scale released period; 6—Access to Scale (agent: Conventional): when accessing the scale viewing screen, the user can choose a month to be exhibited (by default the current month), being that the conventional user can only see future dates when they have been released by the staff coordinator; possibilities of interaction of the users: a) the user can choose a vacant time to register, with a confirmation message coming up, remembering the user that the time responsibility will be his from that moment on, if he confirms his intention of performing that on-duty, being that in having the user confirmation, his name completes the available time of the scale, and an e-mail is sent to staff coordinators informing them; b) one of your times can be made available, being that a message communicating the user that from that moment his time is available to other, but until someone registers, the responsibility is still of the current user and generating an e-mail sent to the staff coordinators informing them about the available time; c) the user can choose a time of another on-duty doctor to application change, with an e-mail to the responsible of the time to be changed being sent, with a link for an area where he can confirm the change or refuse it, being that the response generates a return e-mail to the doctor making the application, informing it if it was accepted or not, being that in case the change has been performed, an e-mail to the staff coordinators will also be generated; 7. Consult on-duties performed by doctor (agent: Coordinator): the coordinator inserts a period and chooses a professional, being exhibited a list of on-duties performed in staff and the total amount; on-duties may be exported to a CSV file.

5. Equipment for implementing a system for managing and coordinating health area staffs that work in an on-duty regime, wherein it predicts the employment of a computer that is known as server (SERV), in which is installed the software (S); the server (SERV), is connected to the internet, that constitutes the virtual environment where the concerned system, indicated by reference (SIS) may be accessed, under many conditions, be it by the Administrator (A), as well as by Coordinator(s) (C) also and still by health area professionals—doctors (P).
